SEQUENCE LISTING (8 sequences)

SEQ ID NO: 1 - DNA, 18 bp, Artificial Sequence
  forward primer for human ACE2 DNA
aggatgtgcgagtggcta

SEQ ID NO: 2 - DNA, 18 bp, Artificial Sequence
  reverse primer for human ACE2 DNA
agggccatcaggatgtcc

SEQ ID NO: 3 - DNA, 24 bp, Artificial Sequence
  forward primer for SARS-CoV-specific subgenomic mRNA5
aggtttcctattcctagcctggat

SEQ ID NO: 4 - DNA, 24 bp, Artificial Sequence
  reverse primer for SARS-CoV-specific subgenomic mRNA5
agagccagaggaaaacaagcttta

SEQ ID NO: 5 - DNA, 20 bp, Artificial Sequence
  detection probe for SARS-CoV-specific subgenomic mRNA5
acctgttccgattagaatag

SEQ ID NO: 6 - DNA, 19 bp, Artificial Sequence
  forward primer for SARS-CoV-specific subgenomic mRNA1
tctgcggatgcatcaacgt

SEQ ID NO: 7 - DNA, 19 bp, Artificial Sequence
  reverse primer for SARS-CoV-specific subgenomic mRNA1
tgtaagacgggctgcactt

SEQ ID NO: 8 - DNA, 17 bp, Artificial Sequence
  detection probe for SARS-CoV-specific subgenomic mRNA1
ccgcaaacccgtttaaa

(19) United States
(12) Patent Application Publication
(10) Pub. No.: US 2009/0083865 A1
(43) Pub. Date: Mar. 26, 2009

(54) Transgenic Mouse Lines Expressing Human Ace2 and Uses Thereof

(76) Inventors: Teh-Sheng Chan, League City, TX (US); Shinji Makino, Galveston, TX (US); Clarence J. Peters, Galveston, TX (US); Chien-Te K. Tseng, Houston, TX (US)

Correspondence Address: Adler & Associates, 8011 Candle Lane, Houston, TX, 77071, US

(21) Appl. No.: 12/087,643

(22) PCT Filed: Jan. 11, 2007
(86) PCT No.: PCT/US2007/000744

§ 371 Date: Jul. 10, 2008

Related U.S. Application Data
(60) Provisional application No. 60/758,189, filed on Jan. 11, 2006.

(51) Int. Cl. A01K 67/027; C12N 15/00

(52) U.S. Cl. 800/3; 435/320.1; 800/18

(57) ABSTRACT

Animal models for severe acute respiratory syndrome-coronavirus infection of humans are needed to elucidate SARS pathogenesis and develop vaccines and antivirals. Transgenic mice were developed expressing human angiotensin-converting enzyme 2, a functional receptor for the virus, under the regulation of a global promoter. A transgenic lineage, designated AC70, was among the best characterized against SARS coronavirus infection, showing weight loss and other clinical manifestations before reaching 100% mortality within 8 days after intranasal infection. Inflammatory mediators were also detected in these tissues, coinciding with high levels of virus replication. In contrast, infected transgene-negative mice survived without showing any clinical illness. The severity of the disease developed in these transgenic mice, AC70 in particular, makes these mouse models valuable not only for evaluating the efficacy of antivirals and vaccines, but also for studying SARS coronavirus pathogenesis and infection by other coronaviruses utilizing human ACE2 for viral entry into cells.

### CROSS-REFERENCE TO RELATED APPLICATION

This U.S. national stage application is filed under 35 U.S.C. §363 and claims benefit of priority under 35 U.S.C. §365 of international application PCT/US2007/000744, filed Jan. 11, 2007, which claims benefit of priority under 35 U.S.C. 119(e) of provisional U.S. Ser. No. 60/758,189, filed Jan. 11, 2006, now abandoned.

### BACKGROUND OF THE INVENTION

1. Field of the Invention

The present invention relates generally to animal models for studying and treating human diseases. More specifically, the present invention provides transgenic mouse lines expressing angiotensin-converting enzyme-2 (ACE2) and their use as human coronavirus infection models for microbiological, immunological, pathological, clinical and epidemiological studies of severe acute respiratory syndrome (SARS) in man and development and testing of antivirals and vaccines for the disease, and as models for infection by other related viruses such as human NL63 virus, which utilize ACE2 for virus entry into host cells.

2. Description of the Related Art

An outbreak of severe acute respiratory syndrome (SARS), caused by the SARS-CoV (coronavirus) a highly transmissible human pathogen, occurred in the fall of 2003. Originating in Guangdong, China, the disease spread rapidly to other parts of Asia and then to the rest of the world. Following the application of intensive public health measures, the disease was successfully contained about 8 months later but not before causing ˜8000 clinical cases with a ˜10% case fatality and tremendous economic impact worldwide.

The most likely hypothesis for the emergence of SARS-CoV is that the virus from the natural reservoir, presumably the Chinese horseshoe bat, Rhinolophus sinicus, adapted to infect civets, which were permissive, and resulted in an epidemic among civets, which were sold in the southern China food markets [19,21]. The virus then spread to humans and underwent further genetic adaptation, particularly to the spike protein to become more efficiently transmissible among the human population [22,35]. It seems unlikely that this first emergence of SARS will be a unique event, because many viruses such as Ebola, Venezuelan equine encephalitis, and epidemic influenza viruses have all returned after a hiatus in transmission. Thus, the need for effective antiviral agents and vaccines would be essential should SARS reemerge in the future. Despite this, there is neither an effective antiviral therapy nor vaccine available to treat SARS.

Animal models are crucial to understanding the pathogenesis of human SARS and developing and evaluating the efficacy of antiviral drugs and vaccines. Although it is known that angiotensin-converting enzyme 2 is a functional receptor for SARS-CoV [20] and that a mouse transgenically expressing human angiotensin-converting enzyme 2 may be a useful animal model of SARS [21] there are no suitable animal models for this disease. None of the several animal models proposed can reproduce human disease including non-human primates (i.e., macaques, African green monkeys, and marmosets), ferrets, hamsters, and mice, including young and aged Balb/c, C57BL/6, and types lacking components of the immune system (i.e., Stat1- and RAG1-knockout mice) [9, 18, 23, 31, 32, 36 and 37]. These animals were shown to be susceptible to SARS-CoV infection and showed viral replication, some degree of histopathology, and, occasionally, limited clinical illness. However, none exhibited consistent clinical illness or mortality. Additionally, all suffer from some disadvantages including high cost, poor availability of reagents, and an immunological response profile to the infecting virus quite unlike that observed in the human disease.

Although the virus infected a few strains of laboratory mice, the infection was of an abortive type associated with no respiratory or systemic symptoms characteristic of SARS and no significant pathological changes in the lungs of the mice. Additionally, the infected mice exhibited no mortality. Thus, the infection of these mice did not mimic human disease. Aged mice, in keeping with elderly humans, have more pathology than do younger normal mice. However, even in the older mice, a mild weight loss has been the only clinical manifestation in response to SARS-CoV infection. Stat1-deficient mice show more pronounced changes than do normal mice, but there is no mortality and the pathological changes are not typical of those found with human SARS. The tropism of coronaviruses is determined primarily by the interaction of the spike (S) protein and the cellular receptors for the virus.

Human angiotensin-converting enzyme 2 (hACE2) has been identified as a major receptor for SARS-CoV. The spike protein of SARS-CoV has a much higher binding affinity to hACE2 than do those of mice, rats, and other animal species, which correlates with much less permissiveness of these animals to this virus [22].

Thus, prior art is deficient in an animal model for SARS that can effectively be used to study infectivity, tissue distribution of SARS-CoV, virus-associated histopathology, inflammatory responses, clinical manifestations, and to test antivirals and vaccines for the disease. The current invention fulfils this long standing need in the art.

### SUMMARY OF THE INVENTION

In one embodiment of the present invention, there is provided an expression vector. Such an expression vector comprises a constitutive promoter, an intron, a polyadenylation site of rabbit β globin and a nucleotide sequence encoding a human angiotensin converting enzyme-2.

In a related embodiment of the present invention, there is provided a transgenic mouse expressing human angiotensin converting enzyme-2 (ACE-2). Such a mouse is derived using the vector described supra.

In another related embodiment of the present invention, there is provided a method of screening for an anti-coronaviral compound. Such a method comprises administering a pharmacologically effective amount of the compound to the transgenic mouse described supra followed by infecting the mouse with the coronavirus. The mouse is then monitored for development of phenotype of the disease caused by the coronavirus, where absence of the development in the presence of the compound indicates that the compound inhibits the binding of the virus to the angiotensin converting enzyme-2 or viral replication and/or maturation subsequent to viral entry, thereby screening for the anti-coronaviral compound.

In yet another related embodiment of the present invention, there is provided a method of screening for a compound that inhibits infectivity of a human coronavirus. This method comprises administering a pharmacologically effective amount of the compound to the transgenic mouse described supra. This transgenic mouse and a control mouse are infected with the human coronavirus. This is followed by comparing the incidence of disease caused by the coronavirus in the mouse subjected to the administration with the incidence of disease in the control mouse lacking the administration, where an absence or a reduced incidence of the disease in the mouse subjected to the administration indicates that the compound inhibits the infectivity of the human coronavirus.

In yet another related embodiment of the present invention, there is provided a method of screening for a vaccine candidate that prevents, or alleviates the symptoms, shortens the course, or reduces the mortality rate, of human corona virus infection.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-1C show the construction and characterization of hACE2 transgene. (FIG. 1A) Diagram of hACE2 expression cassette. The entire open reading frame (ORF) of human ACE2 (hACE2) was amplified by RT-PCR using mRNAs extracted from a human colon cancer cell line, Caco-2. The resulting cDNA of hACE2 was inserted into the expression vector, pCAGGS.MCS, down-stream of the CAG promoter. The resulting plasmid was named pCAGGS.ACE. (FIG. 1B) Western blot analysis of hACE2 expression in transfected human 293 cells. Cell extracts prepared from mock-transfected (Lane 1) or pCAGGS.ACE-transfected human 293 cells (Lanes 2&3) were subjected to Western blot analysis for verifying the transgene expression using monoclonal antibody against ACE2. (FIG. 1C) Tissue expression profile of hACE2 in the transgenic mouse lineages AC70 and AC63. DNA-free RNAs extracted from different organs of transgenic mice at 6-8 weeks of age were subjected to RT-PCR analysis for evaluating the expression of hACE2 mRNA. The RT-PCR products were analyzed on 2% agarose gel. Lanes 1-9 represent spleen, stomach, heart, muscle, brain, kidney, lungs, intestine, and liver, respectively. Data shown are representative of two independently conducted experiments.

FIGS. 2A-2B show weight loss and survival rate of SARS-CoV-infected AC70 Tg⁺ mice and their Tg⁻ littermates. Tg⁺ and Tg⁻ mice at 8-12 weeks of age were intranasally (i.n.) inoculated with 1×10³ TCID₅₀ of SARS-CoV (Urbani strain) in 40 ml saline. Body weights (FIG. 2A) and accumulated mortality (FIG. 2B) of infected Tg⁺ (□) and Tg− (◯) mice were measured and recorded on a daily basis. Weight changes were expressed as the mean percentage changes of infected animals (N=10 per group, including those died) relative to the initial weights at day 0. Error bars represent standard errors.

FIGS. 3A-3D show kinetics of SARS-CoV replication in the lungs and brain of infected mice. Tg⁺ mice (_□) and their Tg⁻ littermates (_◯) (N=15 per group) were inoculated (i.n.) with 10³ TCID₅₀ of SARS-CoV in 40 ml. Three animals in each group were sacrificed daily and virus titers in the lungs and brains were assessed by using both the standard TCID₅₀ assay in Vero E6 cells and quantitative RT-PCR analysis, as described in below. The titers of infectious virus in the lungs (FIG. 3A) and brain (FIG. 3B) were calculated and expressed as log10TCID₅₀ virus per gram of tissue, whereas the relative copy numbers of SARS-CoV mRNA 5 (encoding M protein) of the lung (FIG. 3C) and brain (FIG. 3D) specimens as determined by Q-RT-PCR after normalization against 18S rRNA as the internal control were plotted (the C_{T} method). The average of mRNA5 signals in duplicated samples of individual specimens is depicted. *, p<0.05; ** p<0.01 by a Student's t test, compared between Tg⁺ and Tg⁻ mice.

FIG. 4 shows SARS-CoV replicates in the brains of mice following intra-peritoneal inoculation. Tg⁺ (□) and Tg⁻ (◯) mice, 4 in each group, were inoculated with 10³ TCID₅₀ of SARS-CoV. Tg⁺ mice started to show signs of illness at day 4. Three sick Tg⁺ animals, along with three apparently healthy Tg⁻ counterparts, were sacrificed at day 4 (n=3) and the remaining one from each group was sacrificed at day 5 for titrating infectious virus in the brains. The infectious virus titer of individual mice is expressed as the log₁₀TCID₅₀ per gram of tissue. ** p<0.01 by a Student's t test, compared between Tg⁺ and age-matched Tg⁻ mice.

FIGS. 5A-5J show histopathology and immunohistochemical analysis of SARS-CoV antigen expression in the lungs, brain, and gastrointestinal (GI) tracts of Tg⁺ mice after infection (i.n.). Paraffin-embedded lung (FIGS. 5A to 5G), brain (FIGS. 5H and 5I), and GI tracts (FIG. 5J) sections of infected Tg⁺ mice were analyzed for the pathology and the expression of the nucleocapsid protein of SARS-CoV by the methodologies. SARS-CoV antigen (Red) was readily detectable in the cytoplasm of epithelial cells of the bronchial lining (FIG. 5A) and pulmonary interstitium (FIG. 5B) at day 2. No staining was seen in the same Tg⁺ mouse when immunohistochemistry was performed with normal mouse ascites fluid (FIG. 5C). Serial sections (FIG. 5D, FIG. 5H & FIG. 5E; FIG. 5E, immunohistochemistry) of a bronchus showing intraluminal macrophages and cellular debris in association with viral antigen. Inflammatory cellular infiltrates (arrow) within smooth muscle of a pulmonary blood vessel associated with SARS-CoV antigen (FIG. 5F). SARS-CoV immunostaining of a subepithelial ganglion cell in the lung (FIG. 5G) at day 2. Extensive SARS-CoV antigen expression was first detected on day 3 in large numbers of morphologically intact neuronal and glial cells in the CNS (FIGS. 5H-5I). In the GI tract, the expression of SARS-CoV antigen in ganglia within the subserosal layer (arrow) was detected first at day 4 (FIG. 5J). Magnifications: FIGS. 5A-5F, FIG. 5H, and FIG. 5J, 100X; FIG. 5G, 158X; FIG. 5I, 50X. (FIGS. 5A-5C, FIGS. 5E-5J: napthol red and hematoxylin counterstaining; FIG. 5D: hematoxylin and eosin).

FIGS. 6A-6H show expression of hACE2 in the lungs, brain, and gastrointestinal (GI) tract of Tg⁺ mice. The paraffin-embedded sections of the lungs, brains and GI tract were used to evaluate the expression of the hACE2 by IHC. The hACE2 antigen (red) was readily detectable primarily in the pneumocytes (FIG. 6A), and vascular smooth muscle in the lung (FIG. 6B, arrow). The hACE2 expression in the brain was also abundantly associated with choroid (FIG. 6C), ventricular lining (FIG. 6D), vascular endothelial cells (FIG. 6E), and patches of neuronal and glial elements (FIGS. 6F-6G). Finally, hACE2 was also found in the epithelial lining, muscularis layer, and ganglia of the GI system (FIG. 6H, arrow). Magnification: FIG. 6A, FIG. 6F, and FIG. 6G, 158X; FIG. 6B, FIG. 6D, and FIG. 6H, 50X; FIG. 6C, 25X; FIG. 6E, 100X.

FIGS. 7A-7F show expression of pulmonary cytokines and chemokines IL-1-beta (FIG. 7A), IL-12p70 (FIG. 7B), RANTES (FIG. 7C), IL-12p40 (FIG. 7D), CXCL1/KC (FIG. 7E), and MCP-1 (FIG. 7F) in infected mice. Lung homogenates derived from mice at indicated time intervals after infection (i.n.) were subjected to Bio-Plex analysis for assessing the concentrations of cytokines and chemokines. Among 23 inflammatory mediators tested, the expression of IL-1b, IL-12p40, CXCL1/KC, RANTES, MCP-1, and IL-12p70 was elevated in infected Tg⁺, but not Tg⁻, mice. Duplicated samples of individual specimens were assayed. Data shown are Mean±SEM of infected animals (N=3) at indicated time points. *, p<0.05; ** p<0.01 by a Student's t test, comparing Tg⁺ mice with aged-matched Tg⁻ controls.

FIGS. 8A-8C shows the outcome of SARS-CoV-infected mice of the AC63 line. For the first experiment, Tg⁺ and Tg⁻ mice of the AC63 line, N=10 each, were inoculated (i.n.) with 103 TCID₅₀ of SARS-CoV, and the weight changes were recorded on a daily basis, and expressed as the mean percentage changes of infected animals (FIG. 8A). For the second experiment, ten Tg⁺ AC63 mice were inoculated (i.n.) with 10⁶ TCID₅₀ of SARS-CoV. Five and three infected mice were sacrificed at day 5 and day 8 after infection, respectively, and the titers of infectious virus in the lungs and brains were assessed and expressed as log₁₀/gram (FIG. 8B). The other two infected mice were saved for observing weight changes (FIG. 8C) and other clinical manifestations. ** p<0.01 by a Student's t test, comparing the virus titers between lungs and brain within Tg+ or Tg− mice.

### DETAILED DESCRIPTION OF THE INVENTION

SARS is an emerging infectious disease. The morbidity and mortality due to the disease is unparalleled in the recent history of microbiology. Its impact on a national economy is also enormous. Additionally, it inflicts tremendous damage to human psychology and the societal costs for containing the disease are exceedingly high. Thus, it is imperative to have methods of treatment and prevention of the disease in place before the next epidemic strikes. Hence, the present invention developed a transgenic mouse model for SARS that could be used for the development of antiviral therapeutics and vaccines as well as for conducting studies, which enhanced the understanding of basic science (including microbiological and immunological), clinical and epidemiological aspects of the disease.

Animal models for SARS in well-characterized species that consistently reveal signs of illness, pathological findings, and mortality are highly desirable not only for studying pathogenesis, but also for evaluating the safety and efficacy of antiviral therapeutics and vaccine candidates against SARS-CoV infection. The present invention is directed towards developing a 55 small animal model for SARS using transgenic mice expressing hACE2, the major cellular receptor for SARS-CoV [20]. Not only does this transgenic mouse model support more robust viral growth than its non-transgenic littermates, but it also manifests respiratory and generalized illness, along with tissue pathology and inflammatory cytokine responses. Most significantly, transgenic AC70 mice developed clinical illness, regardless of the route of inoculation, and died uniformly within 8 days after infection, whereas transgenic AC63 mice eventually recovered from the infection, despite the manifestations of clinical illness.

Mice transgenic for hACE2 exhibit distinct clinical courses following SARS-CoV infection which are not seen in infected wild type mice. SARS-CoV infection in the Balb/c and C57BL/6 strains appeared to be short-lived with the viral clearance occurring within 4-8 days after infection. It is noteworthy that these infected wild type strains of mouse did not elicit specific antibody response to SARS-CoV until day 21-28 after infection. Furthermore, mutant mice lacking key immune components, including RAG1^{-/-}, CD1^{-/-}, and bg^{-/-} mice, were shown to clear the infections as efficiently as wild-type mice, suggesting that the classic host anti-viral immune responses might not be critical for resolving SARS-CoV infection in mouse.

Although a prolonged replication of SARS-CoV, accompanied with the onset of clinical illness, was observed in Stat1^{-/-} mice, the patterns of the clinical manifestations appeared to be atypical, in which no evidence of acute inflammatory response in any organ could be observed. Nevertheless, the compromised ability of Stat1-deficient mice to clear virus highlights the importance of innate immunity in controlling SARS-CoV infection in the mouse [9, 12, 36]. Furthermore, as Balb/c mice one year or older of age were more susceptible than younger mice to SARS-CoV, resulting in the development of a limited and non-fatal illness, showing increased pathological changes in the respiratory tract, age is a key determinant of the susceptibility to SARS in animals as in the case for humans [3, 28, 42]. Here again, contrary to the severe and often fatal outcome of SARS in elderly patients, aged mice effectively recovered from the disease, without any mortality. Thus, the transgenic mouse model of the present invention is unique in that it provides defined end-points, including death, weight loss, and respiratory and neurological symptoms as well as virological data and pathological changes, and thus allows for the definitive analysis of the efficacy of antivirals and vaccines to SARS.

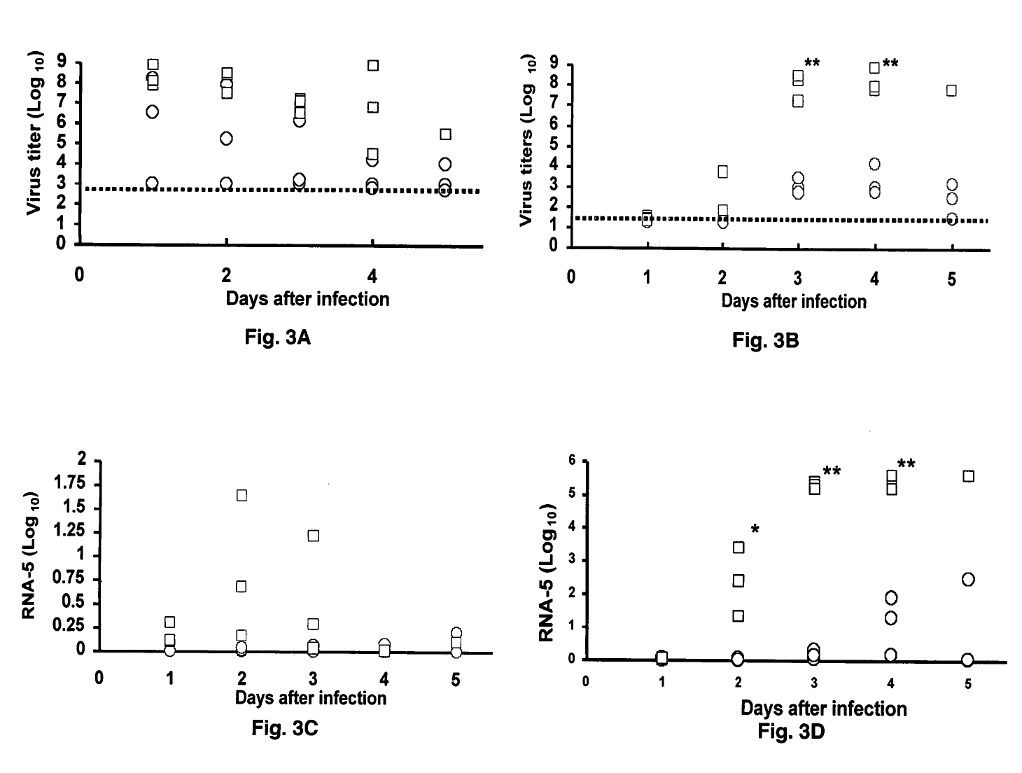

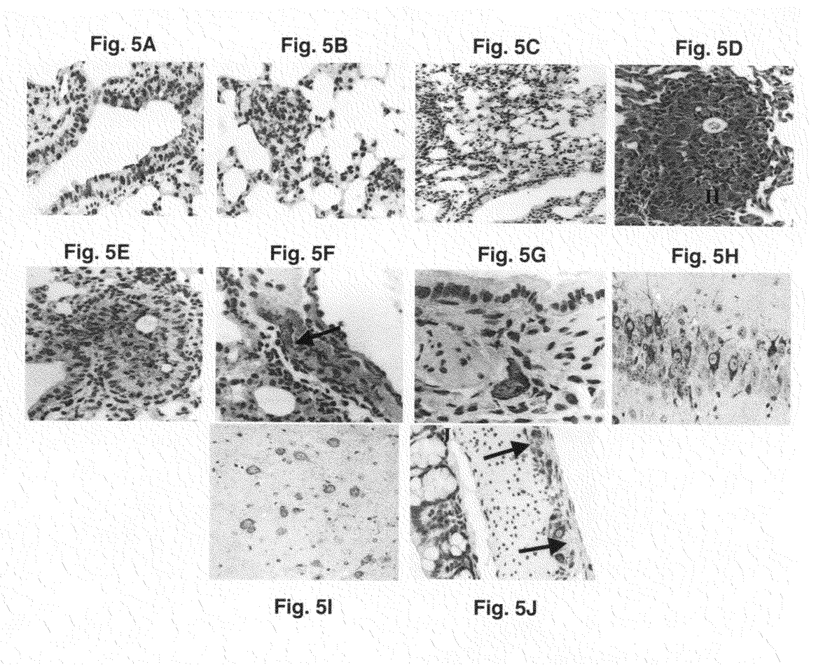

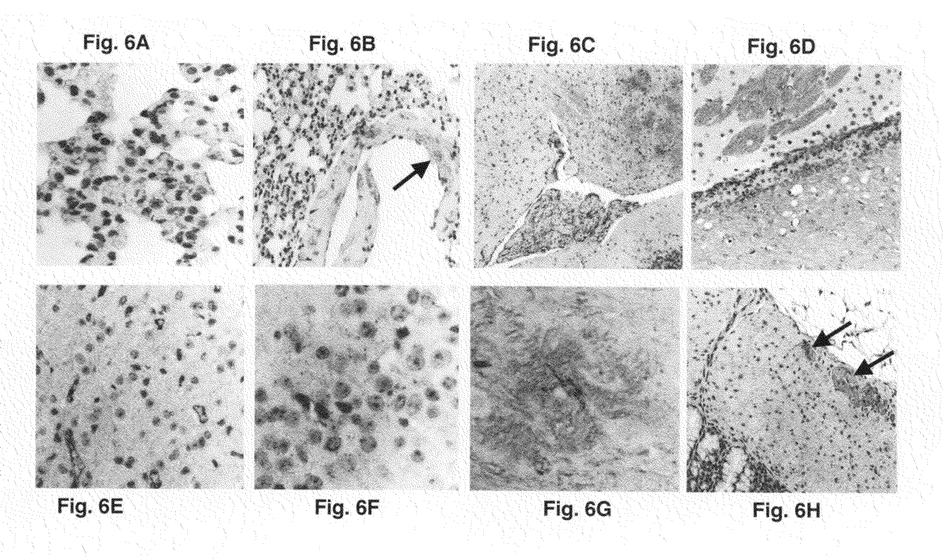

Studies of the kinetics and tissue distribution of viral replication in intranasally (i.n.) challenged AC70 mice demonstrated that the lungs are the major sites of SARS-CoV replication before dissemination to other tissues, particularly the brain (FIGS. 3 & 4). Despite the resemblance in the kinetics of viral replication in the lungs, Tg⁺ mice appear to be more efficient than their Tg⁻ littermates in supporting viral replication, resulting in a more intense pulmonary infection. Virus subsequently spreads from the lungs to the brain of Tg⁺ mice at day 2, and actively replicated there, reaching its maximal level at day 3 and was sustained thereafter until the death of the hosts. The extensive pulmonary and CNS involvement in infected AC70 Tg⁺ mice was confirmed by IHC, which readily revealed the expression of SARS-CoV antigen in patches of pneumocytes and bronchial epithelial cells, as well as in neuronal and glial cells (FIG. 5). Importantly, the expression of viral antigen in the lungs, brain, and GI tracts generally correlated with hACE2 expression (FIGS. 5 & 6). However, whether hACE2 and viral antigen could be detected in the same cells remains unknown. Interestingly, not all hACE2-expressing cells in Tg⁺ AC70 mice were susceptible to SARS-CoV infection. For instance, SARS-CoV infection was not detected in cells lining the endothelium of various organs, despite their intense hACE2-expression (FIG. 6), an observation consistent with the finding with clinical specimens [11,39]. The reason for the lack of SARS-CoV infection in cells highly positive for hACE2 expression in transgenic animals, of the present invention, is not known, but this observation supports the notion that the expression of hACE2 alone might not be sufficient for maintaining effective viral infection [39]. The finding of L-SIGN as another cellular receptor for SARS-CoV [16] might imply that other receptors or co-receptors might be required for viral entry into different cells. It is also possible that surface expression of hACE2 is not present, as shown for Calu-3 cells [41], making those cells insusceptible for SARS-CoV infection. Other host factors, such as pH values, temperature, and oxygen levels, have been implicated in pH-dependent cell entry of poliovirus and rhinovirus [45], and may be also important in defining the tissue tropism of SARS-CoV, which has been shown to undergo pH-dependent cell entry in vitro [27, 49].

SARS is generally recognized as an acute viral pneumonia with the lungs as its main pathological target. However, like other human and animal coronaviruses (CoV), many of which are known to establish acute and persistent infections in neural cells [1, 2, 4, 15], SARS-CoV has been detected by RT-PCR, in situ hybridization, and IHC in the brains and other extra-pulmonary tissues of patients who died of SARS [5, 10, 11, 47]. This neurotropic potential of SARS-CoV is underscored by findings in an experimental mouse model, in which infectious virus was recovered from the brains of infected C57BL/6 mice [9]. Also, several neuronal cell lines of human or rat origins as well as human glioma cell lines are permissive for SARS-CoV replication [48]. Thus, the identification of the brain as a major extra-pulmonary site of SARS-CoV infection, particularly in Tg⁺ mice, falls within the spectrum of coronavirus pathogenesis.

It has been well established that the spread of respiratory viruses to the brain could be mediated either directly through synaptically linked neurons of the olfactory and trigeminal systems, as described in the animals models for Venezuelan equine encephalitis virus (VEE), pseudorabies virus, and avian influenza virus A (H5N1) infection [8, 14, 25, 33], or through the hematogenous route, via the damaged blood-brain barrier. Although the exact route(s) of SARS-CoV dissemination to the CNS remains to be determined, the revelation of low-level viremia in infected (i.n.) Tg⁺ mice at day 2, along with the detection of high virus titers in the brains, but not in the lungs, of intraperitoneally challenged Tg⁺ mice might provide the basis for a hematogenous route of viral transmission.

Autopsy studies have indicated that diffuse alveolar damage (DAD) is the most characteristic pathology in SARS [6, 7, 11, 26]. While SARS-associated diffuse alveolar damage could be caused directly by viral destruction of permissive cells lining the alveoli, the marked heterogeneity of the disease course and the outcome of the infection suggest that host responses may play an important role in the pathogenesis of SARS. Specifically, elevated and prolonged expression of inflammatory mediators, such as CCL2/MIP-1, CXCL8, CXCL9, and CXCL10/IP-10, have been found in SARS patients and experimentally infected (i.n.) C57BL/6 [9, 10, 13, 42, 44, 46]. Although an early enhanced expression of IP-10 has been implicated to be an prognostic indicator for the adverse outcome of SARS-CoV infection [38], the exact protective and/or pathological nature and the spectrum of such exaggerated inflammatory responses in the lungs of SARS patients, especially during the early stages of the infection, has never been explored, as invasive procedures required for such studies were not possible during such an explosive outbreak. Therefore, the robust and highly sustainable SARS-CoV infection in the transgenic mouse model makes it unique to investigate the inflammatory responses within the local tissues, i.e., the lungs and brain.

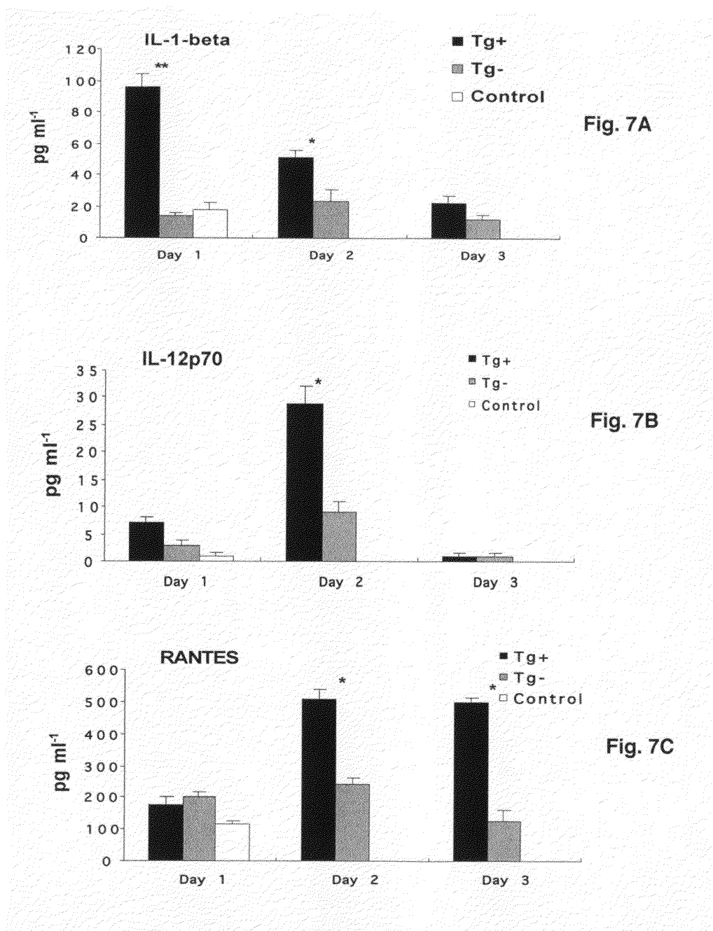

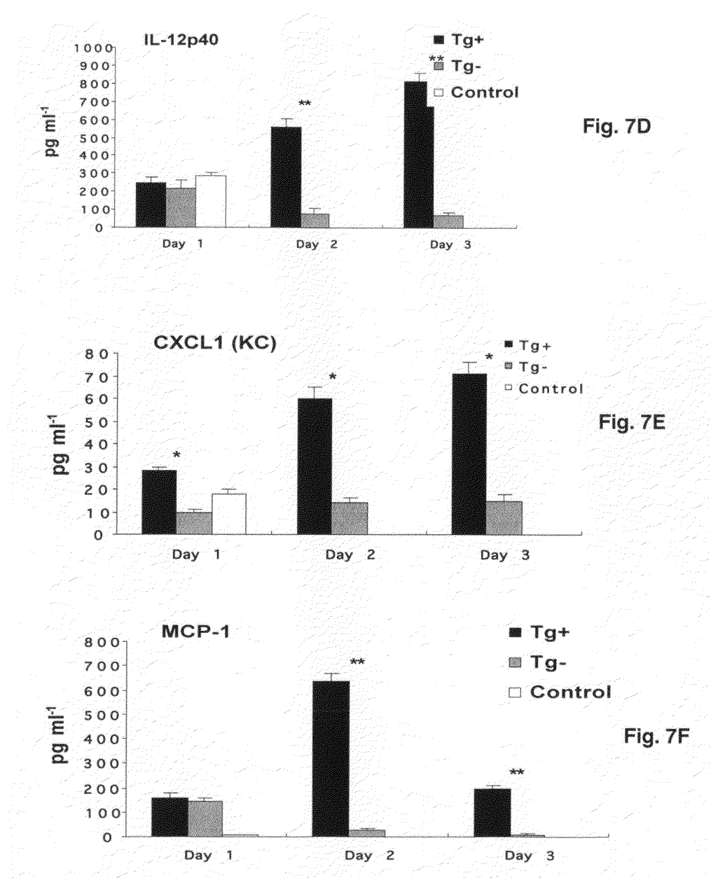

In contrast to Tg⁻ mice that failed to elicit significant inflammatory responses to SARS-CoV infection, infected Tg⁺ mice promptly released elevated levels of IL-1b, IL-12p40, CXCL1/KC, CCL5/RANTES, CCL2/MIP-1, and IL-12p70 within the lungs at days 1 and 2 p.i. (FIG. 7). Although such an acute host inflammatory response did not occur in the brain at day 2, an intense secretion of the aforementioned inflammatory mediators, as well as IL-6, granulocyte-colony stimulation factor (G-CSF), CCL3/MIP-1α, IL-1a, and granulocyte/monocyte colony stimulating factor (GM-CSF) were detected at day 3 (Table 1), concurrently with a marked elevation in the titer of infectious virus.

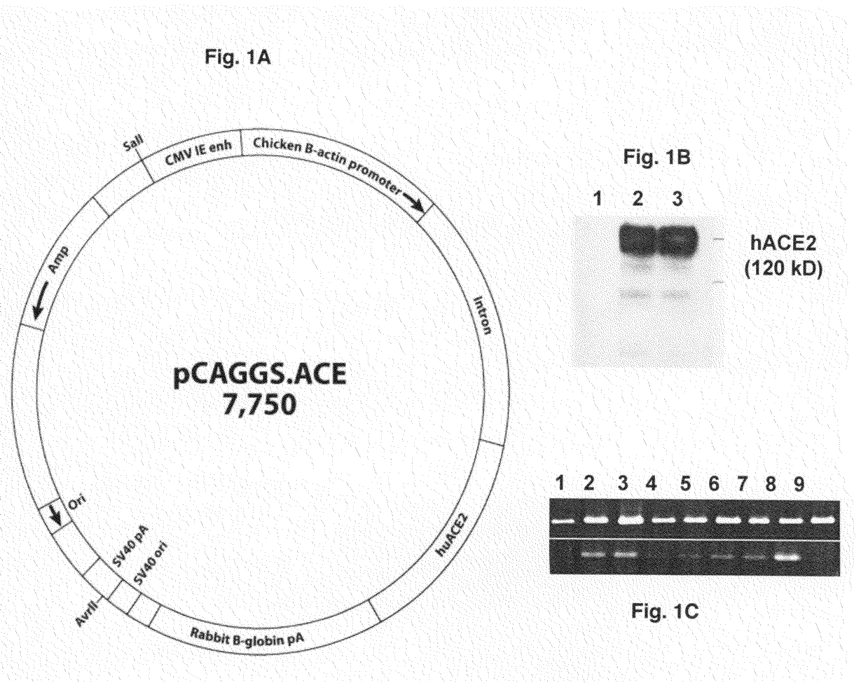

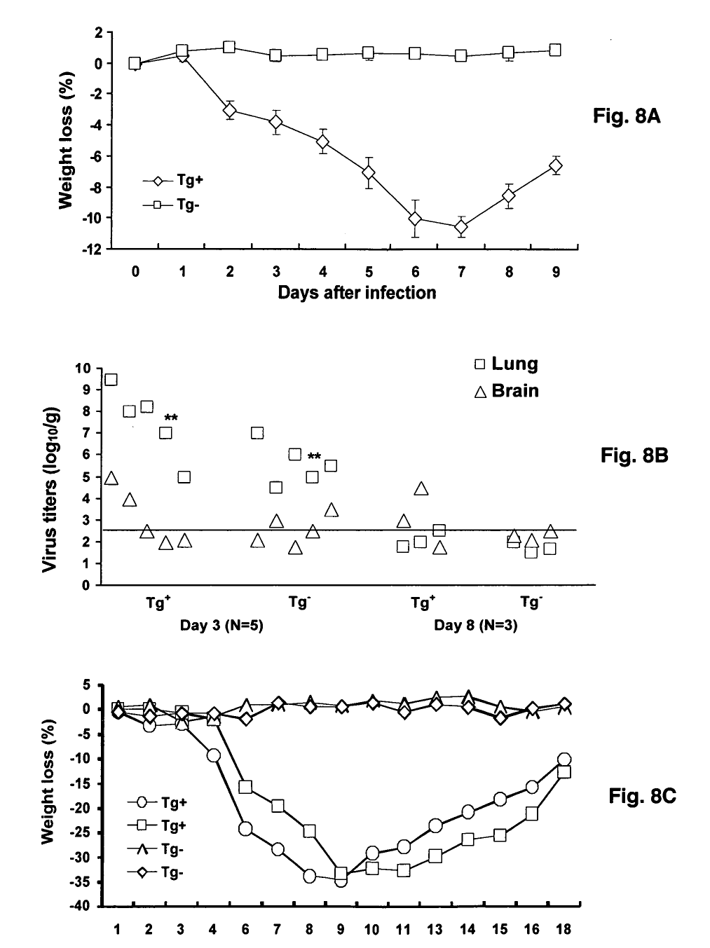

Despite the extensive involvement in the viral infection and the subsequent inflammatory secretion of the CNS, neither necrosis nor cellular infiltrates could be observed in this vital tissue at this stage of the infection. It has been shown that primary cultures of mouse neurons, astrocytes, and microglia were capable of producing innate inflammatory cytokines in response to neurovirulent MHV-JHM infection [30]. Thus, the absence of leukocyte infiltrates in the brains of infected Tg⁺ mice at day 3 might suggest that the resident brain cells are the likely source of these innate inflammatory cytokines. Although the significance of these inflammatory cytokines and chemokines in the pathogenesis of SARS-CoV infection in this transgenic mouse model is currently unknown, some morphologically subtle changes in the CNS of infected Tg⁺ mice may underlie inflammatory cytokines and chemokines-mediated functional derangement of the CNS [29,43], which could be central to the pathogenesis of SARS-CoV infection. Preliminary studies of SARS-CoV infection with mice of the AC63 line indicated that this lineage was also permissive to infection but resistant to the fatal outcome of SARS-CoV infection (FIG. 8). Whether lower hACE2 expression in this AC63 line, compared to AC70 (FIG. 1), could be responsible for their recovery and survival remains to be studied.

In summary, the present invention demonstrates that transgenic mice expressing hACE2 are highly susceptible to SARS-CoV infection, resulting in a wide spectrum of clinical manifestations, including death, depending upon the transgenic lineages. Hence, these transgenic mice will be useful for studying the pathogenesis of SARS and preclinical testing of antiviral agents and vaccine candidates against SARS.

The present invention is directed to an expression vector, comprising a constitutive promoter, an intron, a polyadenylation site of rabbit β globin and a nucleotide sequence encoding a human angiotensin converting enzyme-2. Generally, the constitutive promoter drives global expression of ACE-2. Representative examples of such a promoter includes but is not limited to CAG, CMV, SV40, RSV, PGK or β-actin promoters.

Representative examples of such an expression vector include but are not limited to pCAGGS-ACE.

The present invention is also directed to a promoter which is specifically active in the lungs. Examples of such promoters include but are not limited to lung-specific promoters such as CC10 and SPC promoters and epithelium specific promoters, such as human keratin 10, keratin 14 and keratin 18 promoters.

The present invention is also directed to a transgenic mouse expressing human angiotensin converting enzyme-2 (ACE-2), where the mouse is derived using the above-described vector. Generally, the transgenic mouse with an inbred genetic background is a mouse with a C57BL/6J or a BALB/cJ background. The human ACE-2 is expressed in spleen, stomach, heart, muscle, brain, kidney, lung, liver, intestine or testis of the mouse. Representative examples of such a mouse includes but is not limited to Tg-AC12, Tg-AC22, Tg-AC50, Tg-AC63 or Tg-AC70 mouse. Generally, the Tg-AC70 mouse expresses the transgene abundantly in the lungs and the brains. In addition, the Tg-AC-63 mouse expresses lower levels of the transgene and the expression is restricted to the lungs. Additionally, AC-70 mouse is infected with human coronaviruses. Examples of the coronavirus infecting such a mouse includes but is not limited to severe acute respiratory syndrome causing coronaviral strain (SARS-CoV) or a NL63 strain. In general, such an infection elicits an acute inflammatory cytokine response. Specifically, the cytokine response comprises expression of IL-1b, IL-6, IL-12p40, IL-12p70, G-CSF, CXCL1 (KC), MIP-1α and MCP-1 in the lungs and the brain of infected transgenic mice. Additionally, the expression of the cytokines is delayed and more intense in the brain. Furthermore, the AC-70 mouse infected with the SARS-CoV exhibits the phenotype of severe acute respiratory syndrome (SARS) in humans and dies within 8 days. Additionally, a high virus titer in the lungs and brain is detected in these mice. The phenotype exhibited by such a mouse comprises gross and microscopic abnormalities in the lungs and other organs such as brain, abnormal cardiovascular and renal functions in maintaining electrolyte homeostasis, impaired reproductive functions, high mortality or a combination thereof. Further, the AC-63 mice infected with SARS CoV exhibits the phenotype of severe acute respiratory syndrome (SARS) in humans without any mortality. Specifically, virus titers are observed in the lungs but not in the brain of the infected AC-63 mice.

The present invention is further directed to a method of screening for an anti-coronaviral compound, comprising: administering a pharmacologically effective amount of the compound to the transgenic mouse described supra, infecting the transgenic mouse with the coronavirus; and monitoring the infected mouse for development of phenotype of disease caused by the coronavirus, where absence of the development in presence of the compound indicates that the compound inhibits the binding of the virus to the angiotensin converting enzyme-2, thereby screening for the anti-coronaviral compound.

Examples of the compound inhibiting the binding of the coronavirus to the angiotensin converting enzyme-2 and viral replication include but are not limited to a protease inhibitor, an interferon, a steroid receptor blocking peptide, a siRNA or a natural antiviral compound. Such a compound may be administered by any route known to a person having ordinary skill in this art, e.g., oral, intravenous, intranasal or inhalational. Examples of the coronavirus infecting such a mouse includes but is not limited to severe acute respiratory syndrome causing coronaviral strain (SARS-CoV) or a NL63 strain.

The present invention is further directed to a method of screening for a compound that inhibits infectivity of a human coronavirus, comprising: administering a pharmacologically effective amount of the compound to the transgenic mouse described supra, infecting the transgenic mouse and a control transgenic mouse with the human coronavirus, and comparing the incidence of disease caused by the human coronavirus in the mouse subjected to the administration with the incidence of disease in the control mouse lacking the administration, where an absence or a reduced incidence of the disease in the mouse subjected to the administration indicates that the compound inhibits the infectivity of the human coronavirus.

Generally, the compound inhibits the infectivity by inhibiting the binding of the human coronavirus to angiotensin converting enzyme-2, by eliciting a protective response against the human coronavirus or a combination thereof. Examples of the compound inhibiting the binding of the coronavirus to the angiotensin converting enzyme-2 includes but is not limited to a peptide that blocks receptor binding of the virus. Such a compound may be administered by any route known to a person having ordinary skill in this art, e.g., oral, intravenous, intramuscular or subcutaneous.

Additionally, the compound or reagent eliciting the protective immune response against the human coronavirus is an immunogenic compound effective as a vaccine. Examples of such a compound or reagent includes but is not limited to the one that comprises a viral antigen, a peptide, a viral-like particle, an inactivated virus, a live attenuated virus or a viral DNA. Such a compound or reagent is administered intramuscularly, intranasally or percutaneously. Additionally, parameters for the analysis of vaccine efficacy include prevention of disease, alleviation of symptoms and shortening of the disease course and reduction of mortality rate.

Generally, the human coronavirus whose infectivity is inhibited is a coronavirus that uses human angiotensin converting enzyme-2 as a receptor for entry. Examples of such coronaviruses include but is not limited to SARS-causing coronaviral strain or a NL63 strain. Additionally, the compound is administered concurrent with or prior to the infection of the mouse with the coronavirus.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

### Construction and the Expression of the hACE2 Transgene

The cDNA coding for hACE2 was generated by RT-PCR amplification from a human colon carcinoma cell line, Caco2, which supported SARS-CoV replication [24]. The resulting PCR product was cloned into the pSTblue-1 cloning vector (Novagen) and the entire region corresponding to the ACE2-gene was confirmed by sequencing. The cDNA fragment containing ACE2 sequences was subsequently cloned into a eukaryotic expression vector, pCAGGS/MCS (from Dr. Yoshihiro Kawaoka, University of Wisconsin at Madison), under the control of the CAG promoter, a composite promoter consisting of the CMV-IE enhancer and the chicken β-actin promoter, and containing the rabbit globin splicing and polyadenylation site. To verify the expression of hACE2, human embryonic kidney 293 cells were transfected with the resulting plasmid construct, designated pCAGGS-ACE2 (FIG. 1A), using Lipofectamine 2000 reagent (Invitrogen, Carlsbad, Calif.) per the manufacturer's protocols. Cell extracts were prepared at 24 hrs after transfection, and the expression of hACE2 was examined by Western blot analysis using polyclonal antibody against hACE2 (R&D system).

### EXAMPLE 2

### Generation and Characterization of Transgenic Mouse

Transgenic mice expressing human ACE2 were generated by microinjecting the expression cassette, which was excised from pCAGGS-ACE2 by AvrII/SalI digestion and purified by agarose gel electrophoresis, into pronuclei of zygotes from the intercross of (C57BL/6J×C3H/HeJ) F1 parents. Transgenic mice were initially identified by PCR of genomic DNA with hACE2-specific primers: forward 5′-AGG ATG TGC GAG TGG CTA-3′ (SEQ ID NO. 1) and reverse 5′-AGG GCC ATC AGG ATG TCC-3′ (SEQ ID NO. 2), amplifying a transgene-specific fragment of 195 bp (data not shown). A total of five lineages, expressing different levels of hACE2 in the tail biopsies, were established. Two of the lineages, designated AC70 and AC63, respectively, were investigated with regard to the tissue distribution of hACE2 transgene expression by RT-PCR with the same hACE2-specific primers as above, followed by agarose gel analysis of PCR products.

### EXAMPLE 3

### Virus and Cells

The Urbani strain of SARS-CoV at the Vero 2^{nd} passage level, provided to us by Dr. T. G. Ksiazek, Centers for Disease Control and Prevention (Atlanta, Ga.), was used. Vero E6 cells (American Type Culture Collection) were used to grow virus stocks and as indicator cells for the virus infectivity assay. Stocks of SARS-CoV were prepared by passaging them twice in Vero E6 cells at a low MOI (0.001), generating cell-free viral stocks with titers expressed as a 50% tissue culture infectious dose (TCID₅₀)/ml sample (typically, 1×10⁸ TCID₅O/ml), aliquoted and stored at −80° C. All experiments involving infectious virus were conducted at the University of Texas Medical Branch (Galveston, Tex.) in approved biosafety level 3 laboratories and animal facilities, with routine medical monitoring of staff.

### EXAMPLE 4

### Viral Infection and Morbidity and Mortality Studies of Infected Mice

All animal experiments were carried out in accordance with animal protocols approved by the IACUC committee at UTMB. Mice used in this study were backcrossed 2-3 times onto either C57BL/6 or Balb/c background. No difference with regard to the susceptibility to SARS-CoV was observed among mice derived from the different genetic background. Briefly, anesthetized transgenic mice and their non-transgenic littermates at the ages of 8-20 weeks were inoculated, via the intranasal (i.n.) route, with 10³ or 2×10⁵ TCID₅₀ of virus in 40 ml saline. Animals were weighed and observed daily for sign of illness and mortality. In some experiments, infected mice were sacrificed at indicated time intervals after inoculation to obtain selected tissue specimens to define viral distribution by viral titration in Vero E6 cells and by quantitative RT-PCR assay and for histopathology analysis.

### EXAMPLE 5

### Assessment of Tissue Distribution of SARS-CoV in Infected Animals

In addition to blood, throat and nasal turbinate washes, and urine, solid tissue specimens (i.e., the lungs, brain, heart, liver, kidney, spleen, mesenteric lymph nodes (mLNs), small and large bowels, and feces were weighed and homogenized in a PBS/10% FCS solution using the Tissue Lyser-Qiagen (Retsch, Haan, Germany) to yield 10% tissue/PBS suspensions. These suspensions were clarified by centrifugation and subjected to virus titration with the standard infectivity assay using Vero E cells. The virus titer of individual samples was expressed as TCID₅₀ per ml or per gram of sample.

### EXAMPLE 6

### Quantitative Real-Time (O-RT)-PCR for SARS-CoV Subgenomic RNAs

Total RNA was isolated from tissues of infected mice at indicated time intervals after infection using an RNeasy Mini Kit (Qiagen Sciences). Contaminating genomic DNA was removed upon digestion with DNase I during the extraction procedure. Resulting RNA specimens were subjected to one-step Q-RT-PCR analysis for assessing the expression of SARS-CoV-specific subgenomic mRNA1 and mRNA5, according to the methodologies established in our laboratories [40,41]. The following primers and detection probes were used: for RNA 5: forward 5′-AGG TTT CCT ATT CCT AGC CTG GAT T-3′ (SEQ ID NO. 3), reverse 5′-AGA GCC AGA GGA AAA CAA GCT TTA T-3′ (SEQ ID NO. 4), and the sequence of ACC TGT TCC GAT TAG AAT AG (SEQ ID NO. 5), as a detection probe; and for RNA 1: forward 5′-TCTGCG GAT GCA TCA ACG T-3′ (SEQ ID NO. 6), reverse 5′-TGT AAG ACG GGC TGC ACT T-3′ (SEQ ID NO. 7), and the sequence of CCG CAA ACC CGT TTA AA (SEQ ID NO. 8), as a detection probe, all of which were derived by using the Assays-by-Design software (Applied Biosystems). The selected primer set and Taq-Man probe for 18S rRNA were used as the endogenous control. Briefly, 80 ng RNA was transferred to separate tubes for amplifying the target genes and endogenous control (18S rRNA), respectively, by using a TaqMan one-step RT-PCR master mix reagent kit. The cycling parameters for one-step RT-PCR were: reverse transcription at 48° C. for 30 min, AmpliTaq activation at 95° C. for 10 min, denaturation at 95° C. for 15 sec and annealing/extension at 60° C. for 1 min. A total of 40 cycles was performed on an ABI PRISM 7000 real-time thermocycler (Applied Biosystems) following the manufacturer's instructions. DNA fragments encoding target genes were amplified in triplicate and relative mRNA levels for each sample were calculated as follows: A cycle threshold (ΔC_{T})=C_{T} target genes−C_{T} 18S rRNA. The relative abundance of the RNA for hACE2 or for SARS-CoV was expressed as 2⁻⁽^{_{Δ}}^{Ct infected-}^{_{Δ}}^{Ct mock)}.

### EXAMPLE 7

### Histopatholgical and Immunohistochemical Studies

The brain (day 2 post-infection) and lungs (day 3 post-infection) of SARS-CoV-infected AC70 were fixed in formalin and embedded in paraffin. The paraffin sections were stained with antibodies for SARS-CoV antigen and counterstained with hematoxylin.

The viral antigens were present in the bronchial epithelial cells and the interstitial cells of the lungs, indicating viral replication in many cell types of this organ (FIGS. 5A-5B). There was mild cellular infiltration in the alveolar septa causing narrowing but not obliteration of the air space. In the brain, viral antigens were detected in many neurons and a few glial cells but there was a little evidence of inflammation (FIG. 5H-5I). In view of these histological pictures and the clinical signs, it was likely that the infected ACE2 transgenic mice did not die from pulmonary insufficiency but rather from neurological disorder.

### EXAMPLE 8

### Measurement of Inflammatory Cytokines and Chemokines

Inactivated (g-irradiation) tissue homogenates were used to define cytokine profiles by the Bio-Plex Cytometric Bead Array (Bio-Rad, Hercules, Calif.) analysis, according to the manufacturer's recommendation. This technology was used to simultaneously quantify up to 23 inflammatory mediators.

### EXAMPLE 9

### Statistical Analysis

Viral titers and the contents of inflammatory cytokines and chemokines were compared between groups of mice and tested for significance in differences by Student's t test.

### EXAMPLE 10

### Generation of hACE2 Transgenic Mice and Detection of Transgene Expression

To verify hACE2 expression of pCAGGS-ACE2 plasmid (FIG. 1A), human embryonic 293 cells were transfected with this plasmid and cells extracts were prepared at 24 h post transfection. The expression of ACE2 was examined by Western blot analysis using specific antibody to ACE2. As shown in FIG. 1B, an abundant expression of ACE2 of about 120 kD was readily detectable in transfected cells, whereas this signal could not be detected in untransfected cells.

Microinjection of this ACE2-expressing cassette DNA into F2 zygotes from F1 mice (C57BL/6J×C3H/HeJ) resulted in five viable founder animals, designated AC-12, -22, -50, -63, and -70, respectively. These founders were backcrossed to C56BL/6 or Balb/c mice. The hACE2 transgene in the litters was monitored by PCR, showing that all the founders appeared to transmit the transgene to their progenies. The hACE2 expression in different organs of AC70 and AC63 transgenic lineages was subsequently evaluated by RT-PCR. As shown in FIG. 1C, the ACE2 transgene was ubiquitously expressed in both AC70 and AC63 lines, with AC70 at a much higher level than AC63.

### EXAMPLE 11

### SARS-CoV-Induced Morbidity and Mortality in Tg⁺ AC70 Mice

High levels of ACE2 expression in AC70 mice prompted us to investigate the outcome of SARS-CoV infection in this particular lineage. The susceptibility of transgene-positive (Tg⁺) and their transgene-negative (Tg⁻) littermates, ranging from 2- to 6-months of age, was determined in a pilot study by inoculating mice with either 2×10⁵ or 10³ TCID₅₀ of SARS-CoV per mouse, via the i.n. route. All infected Tg⁺ mice, but not their age-matched Tg⁻ littermates, developed an acute wasting syndrome and died within 4 to 8 days post infection (pi) with either dose. Thus, the lower dosage, i.e., 10³ TCID₅₀, was adopted in the subsequent studies to verify the pathogenesis of SARS-CoV infection.

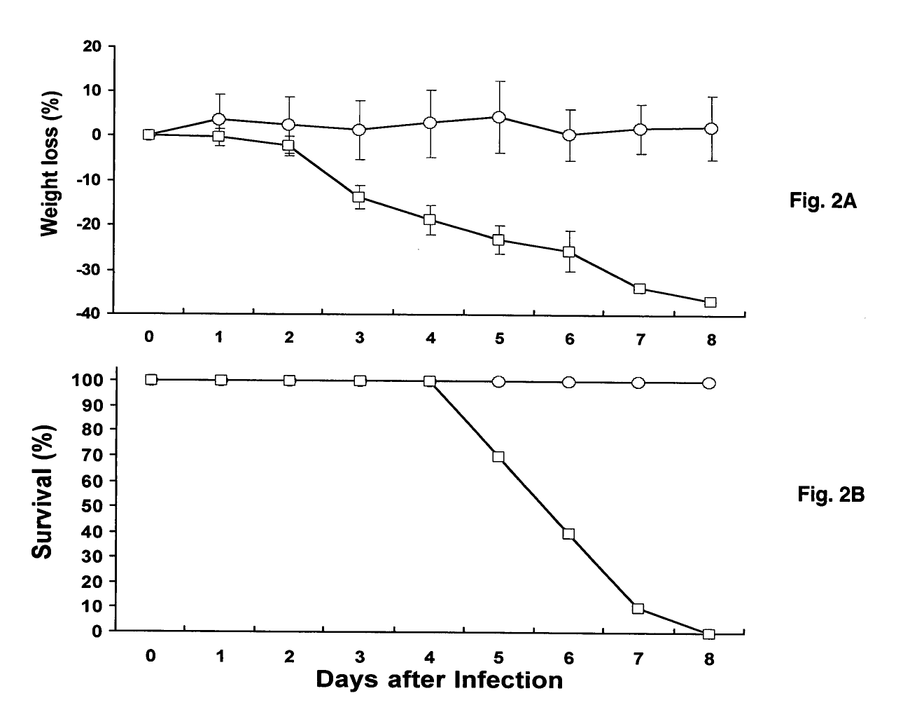

For the next experiment, Tg⁺ and Tg⁻ AC70 mice were inoculated (i.n.), 10 animals in each group, with SARS-CoV. Infected mice were observed for signs of clinical illness daily. Early clinical manifestations of infected Tg⁺ mice included ruffled fur, lethargy, and rapid, shallow breathing, accompanied by the persistent weight loss, which could reach up to 35-40% in some mice (FIG. 2A). This relentless weight loss may have been caused by wastage, which is associated with many viral diseases and aggravated by inappetance, due to the decreased and apparently uncontrolled directional movement. There were no seizures or obvious paralysis, but they died after a period of immobility lasting 1-3 days. Mortality began on day 4 p.i. and reached 100% by day 8 μl (FIG. 2B). All of the infected Tg⁻ mice continued to thrive throughout the entire course of infection without any significant weight loss or other clinical manifestations, findings which correlated with previous reports of normal mice [9,36].

### EXAMPLE 12

### Distribution of SARS-CoV in Tissues of Infected AC70 Mice

The kinetics and tissue distribution of infectious virus next investigated by inoculating (i.n.) age-matched Tg⁺ and Tg⁻ mice, 15 per group. Three mice in each group were sacrificed at daily intervals, except for the fifth day, at which only one Tg⁺ mouse survived the infection, and the titers of infectious virus in various tissues were determined in Vero E6 cells. Among the tissues examined, the lungs and the brain were the major sites of viral replication, particularly in Tg⁺ mice. As shown in FIG. 3A, maximum viral titers were detected in the lungs within 1-2 days p.i. with a median of 10^{8.5} and 10^{6.5} TCID₅₀ per gram (TCID₅O/g) of tissue for Tg⁺ and Tg⁻ mice, respectively. Although the viral titers gradually decreased thereafter in the lungs of both strains of mice, relatively higher virus titers were recovered from Tg⁺ mice than from their Tg⁻ littermates during the entire course of infection.

Viral replication was also detected in the brain of infected mice, with strikingly different kinetics from that of the lungs. A low-titer of the virus was first detected in the brain of Tg⁺ mouse on day 2 p.i. Thereafter, virus replication proceeded rapidly and reached a median of more than 10⁸ TCID₅₀/g at day 3 p.i. (FIG. 3B). In contrast to the decreasing trend of infectious virus over time in the lungs, viral titers remained high in the brain, starting at day 3 until the death of the host. Although infectious virus was also detectable in the brain of some Tg⁻ mice at day 3 p.i., the titers of virus were significantly lower (p<0.01) than those of Tg⁺ mice. The kinetics of SARS-CoV replication in both tissues was confirmed by Q-RT-PCR analysis targeting SARS-CoV-specific sub-genomic mRNA5 (FIGS. 3C and 3D) and mRNA1.

A low, but detectable, level of infectious virus, usually less than 10⁴ TCID₅₀/ml or g was also detected in 8 out of 12 ( 8/12) Tg⁺ and 4/15 Tg⁻ nasal washes, 3/12 Tg⁺ and 1/15 Tg⁻ liver specimens, and 1/12 Tg⁺ large bowels collected from infected animals at various time points. However, there was no detection of infectious virus in throat swabs, blood, heart, spleen, mLNs, kidneys, urine, or feces by the infectivity assay, in which the detection limit was greater than 10³ TCID₅₀/ml or g of tissues.

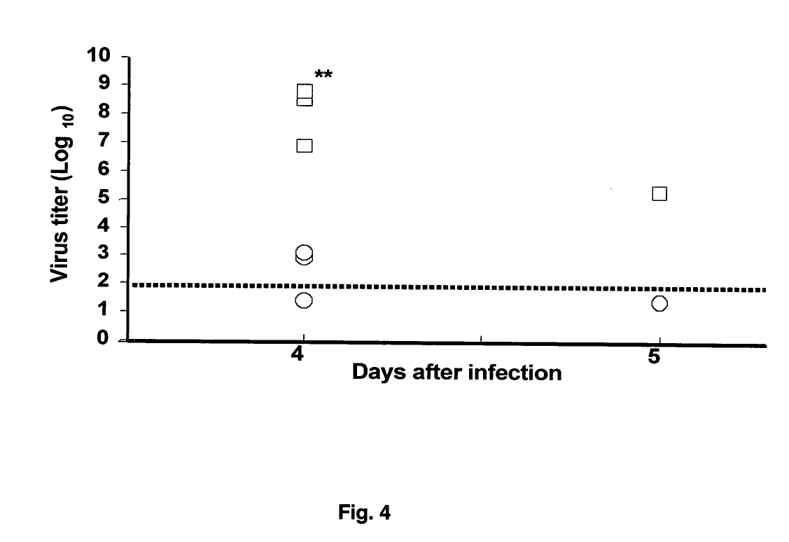

To investigate whether the virus spread to the brain was unique to the i.n. route of infection, AC70 mice were challenged with 103 TCID₅₀ of virus through the intra-peritoneal (i.p.) route. While infected Tg⁻ mice appeared to be healthy, Tg⁺ animals started to show signs of illness at day 4 p.i., and were thus sacrificed, along with four apparently “healthy” Tg⁻ littermates, for determining the viral titers in the lungs and the brain. As shown in FIG. 4, all infected Tg⁺ mice exhibited high viral titers in the brains, whereas only two of the four infected Tg⁻ mice had infectious virus in this organ at a significantly lower titer (p<0.01). Interestingly, there was no recovery of infectious virus from the lungs of either strain of mice. These results clearly indicated that the dissemination of infectious SARS-CoV to the brain is independent of the route of the infection.

Although infectious virus was detected in the circulation in the earlier studies, the extremely high recovery of infectious virus from the brains of i.p. challenged Tg⁺ mice prompted re-evaluation of the viremic status of infected animals. Five Tg⁺ mice were inoculated (i.n.) with 10³ TCID₅₀ of the virus. To increase the sensitivity of detection, instead of using the blood specimens that were diluted (1:10) and a small portion of the spleen in earlier studies, undiluted blood specimens were collected and the whole spleens of infected animals at day 2 p.i. for the infectivity assays. With this improved method, infectious virus was detected from both tissues in all of the infected animals at a titer ranging from 10² to 10^{2.5} TCID₅₀, a titer that was below the limit of detection in the earlier studies, suggesting that a low-level of viremia did exist in infected Tg⁺ mice.

### EXAMPLE 13

### Histopathology and Immunohistochemistry

The histopathology of SARS is characterized by an interstitial pneumonitis, diffuse alveolar damage, with extensive alveolar collapse and filling of remaining alveoli with fluid and desquamated epithelial cells [17, 26, 34]. Histological examination of infected AC70 mice at day 2 p.i. revealed a moderate interstitial pneumonitis with focal thickening of alveolar wall, and filling of alveolar sacs and small airways with cellular debris and macrophage-like cells. Immunohistochemical (IHC) staining showed that SARS-CoV antigen was readily detected in the bronchial epithelial cells and in association with the inflammatory infiltrate in the pulmonary interstitium of infected Tg⁺ mice (FIGS. 5A-5B). This detection of SARS-CoV antigen was specific, since staining of the lung tissues obtained from the same infected animals with irrelevant mouse antibodies was negative for the viral antigen (FIG. 5C). Infected bronchial epithelial cells showed cytoplasmic swelling and blebbing, and were surrounded by moderate inflammatory mononuclear infiltrates. Cellular debris associated with abundant viral antigen was seen within the bronchial lumen (FIGS. 5D-5E). Although the SARS-CoV was also detected in the lungs of infected Tg⁻ mice, the frequency of infected cells and viral antigen was much lower than those of Tg⁺ mice (data not shown). No extrapulmonary SARS-CoV antigens were detected by IHC in Tg⁻ mice. SARS-CoV antigen was also detected in vascular smooth muscle and ganglion cells in the lungs of Tg⁺ mice by day 3 (FIGS. 5F-5G). SARS-CoV antigen present in the smooth muscle of blood vessels was associated with a mild to moderate vasculitis (FIG. 5F).

High levels of SARS-CoV antigen expression were also detected at days 3 and 4 p.i. in abundant neurons and glial cells of the CNS of infected Tg⁺ mice (FIGS. 5H-5I), consistent with high titers of the virus in the brain at this stage. However, no necrosis or inflammatory reaction could be seen in association with the presence of SARS-CoV antigen in the CNS. Additionally, viral antigen was detected in the GI tracts of Tg⁺ mice at day 6 p.i., even though infectious virus was rarely recovered in this tissue. Within the GI tract, virus antigen was restricted to the subserosal ganglia (FIG. 5J) and smooth muscle of the intestinal wall.

The expression of hACE2 antigen was detected in the lungs, kidneys, liver, heart, skeletal muscle, spleen, LN, pancreas, gastrointestinal smooth muscle and ganglia, vascular endothelium, adrenal, and CNS of Tg⁺ mice. In the IHC assay, staining of hACE2 was specific for the human protein; no such expression was detected in Tg⁻ mice and no staining was seen using normal goat serum as a negative control. Although two-color staining was not performed to co-localize hACE2 and viral antigen expression, in the lungs and GI, the viral distribution correlated well with the pattern of expression observed for hACE2. In the lungs of Tg⁺ mice, hACE2 was detected primarily in the pneumocytes, vascular smooth muscle, and ganglion cells (FIGS. 6A-B). Expression was found focally in the muscularis, and subserosal ganglia of the GI system, in similar areas to those where SARS-CoV antigen was present (FIG. 6H). In comparison, in the CNS the distribution of viral antigen and hACE2 was significantly different. High levels of hACE2 expression were detected in choroid, ventricular lining, and vascular endothelial cells, while only rare neurons and glial cells showed minimal expression of hACE2 (FIGS. 6C-6G). However, intense staining of SARS-CoV was only detected in neuron and glial cells (above), suggesting that not all of the hACE2-expressing cells are susceptible to the infection.

### EXAMPLE 14

### SARS-CoV-Induced Cytokines and Chemokines in the Lungs and Brains of Mice

The mechanism of SARS-associated lung pathology remains unknown. However, pathological studies with postmortem specimens of SARS patients reveal diffuse alveolar damage (DAD), hemophagocytosis, and prominent infiltration of activated macrophages (MF) in the lungs, which suggests that an intense and un-regulated inflammatory response within the lungs may be partially responsible for the pathogenesis of human SARS-CoV infection [26].

The severity of the disease developed in Tg⁺ mice in response to SARS-CoV infection prompted study of the host responses by measuring the contents of various inflammatory mediators in the lungs and brain, two of the most affected tissues. As shown in FIGS. 7A-7F, among 23 inflammatory mediators measured, negligible levels of inflammatory cytokines were detected in the lungs of infected Tg⁻ mice over time, compared to the levels in uninfected controls, suggesting that SARS-CoV infection failed to induce cytokine production in Tg⁻ mice. In contrast, elevated levels of IL-1beta (FIG. 7A), IL-12p70 (FIG. 7B), RANTES (FIG. 7C), IL-12ₚ₄₀ (FIG. 7D), CXCL1 (KC) (FIG. 7E), and MCP-1 (FIG. 7F) expression were readily detected in the lungs of Tg⁺ mice in at least one time point during the first three days of the infection. In contrast, elevated levels of IL-1b, IL-12ₚ₄₀, IL-12ₚ₇₀, CXCL1 (KC), RANTES, and MCP-1 expression were readily detected in the lungs of Tg⁺ mice in at least one time point during the first three days of the infection. The SARS-CoV-induced inflammatory response in the brain was similarly evaluated. There was no significant expression of inflammatory mediators in infected Tg⁺ and Tg⁻ mice on both day 1 and day 2. However, highly elevated levels of IL-6, IL-12ₚ₄₀, G-CSF, CXCL1 (KC), MIP-1α, and MCP-1 were detected at day 3 in the brains of Tg⁺ mice, but not their Tg⁻ littermates (Table 1). Additionally, the secretion of IL-1a, IL-1b, GM-CSF, IL-12ₚ₇₀, and RANTES was increased to varying extents in the brain of the Tg⁺ mouse. Other inflammatory mediators, such as IL-2, IL-3, IL-4, IL-5, IL-9, IL-10, IL-13, IL-17, IFN-g, and TNF-a, were not significantly induced.

Taken together, these results clearly demonstrate that Tg⁺ mice, which had much higher levels of SARS-CoV replication in the lungs and brain than their Tg⁻ littermates, as shown in FIG. 3, could promptly elicit a strong inflammatory cytokine reaction to acute SARS-CoV infection. Importantly, this acute inflammatory response occurred later and more intensely in the brain than in the lungs, consistent with the level of virus replication in respective organs.

### EXAMPLE 15

### SARS-CoV Infection in Transgenic AC63 Mice

The susceptibility of Tg+AC63 mice to SARS-CoV infection was initially evaluated by using the same challenge strategy, i.e., 103 TCID₅₀ of SARS-CoV, via the i.n. route. The Tg⁺ AC63 mice were more susceptible to SARS-CoVinfection than their Tg-littermates, as evidenced by a moderate but progressive weight loss until day 8 (FIG. 8A). However, in contrast to the uniform mortality of infected Tg+AC70 mice, all infected AC63 mice eventually recovered from the weight loss without any death. The tissue distribution of infectious virus was next investigated as well as whether inoculation with a higher dose of virus could result in a fatal outcome. Both AC63 mice and control littermates were inoculated (i.n.), 10 of each, with 106 TCID₅₀ of virus. For quantifying the viral loads in the lungs and brain, five and three mice from each group were sacrificed at days 3 and 8, respectively. The remaining animals were kept for assessing the morbidity and mortality. Infected Tg⁺, but not Tg⁻, mice started to show an progressive weight loss, along with other clinical manifestations, between day 3 and day 4.

As shown in FIG. 8B, Tg⁺ mice appeared to be more susceptible to SARS-CoV infection than their Tg⁻ littermates, as evidenced by a much higher titer of infectious virus in the lungs. Additionally, 4 out of 5 Tg⁺ mice had high virus titers in the lungs, whereas only 2 had low-to-moderate titers in the brain at day 3. Infectious virus was no longer detectable in the lungs at day 8, even though one animal still had detectable virus in the brain. Remarkably, despite the severity of the illness, as evidenced by the profound weight loss, the remaining animals started to show signs of recovery between day 8 and day 9, regained some of the lost weight in one week thereafter (FIG. 8C), and recovered completely in ensuing one month when the experiment was terminated.

The following references were cited herein:
1. Arbour, et al., Talbot. 1999. J. Virol. 73:3338-3350.
2. Butler, et al., 2006. Virology 347:410-421.
3. Chan, et al., 2003. Respirology. 8 Suppl:S36-S40.
4. Coley, et al., 2005. J. Virol. 79:3097-3106.
5. Ding, et al., 2004. J. Pathol. 203:622-630.
6. Ding, et al., 2003 J. Pathol. 200:282-289.
7. Franks, et al., 2003. Hum. Pathol. 34:743-748.
8. Gerdts, et al., 2000. J. Virol. 74:817-827.
9. Glass, et al., 2004. J. Immunol. 173:4030-4039.
10. Gu, et al., 2005. J. Exp. Med. 202:415-424.
11. Hamming, et al., 2004. J. Pathol. 203:631-637.
12. Hogan, et al., 2004. J. Virol. 78:11416-11421.
13. Huang, et al., 2005. J. Med. Virol. 75:185-194.
14. Iwasaki, et al., 2004. Acta Neuropathol. (Berl) 108:485-492.
15. Jacomy, et al., 2006. Virology 349:335-346.
16. Jeffers, et al., 2004. Proc. Natl. Acad. Sci. U.S. A 101:15748-15753.
17. Ksiazek, et al., 2003. N. Engl. J. Med. 348:1953-1966.
18. Kuiken, et al., 2003. Lancet 362:263-270.
19. Lau, et al., 2005. Proc. Natl. Acad. Sci. U.S. A 102:14040-14045.
20. Li, et al., 2003. Nature 426:450-454.
21. Li, et al., 2005. Science 310:676-679.
22. Li, et al., 2006. J. Virol. 80:4211-4219.
23. Martina, et al., 2003. Nature 425:915.
24. Mossel, et al., 2005. J. Virol. 79:3846-3850.
25. Mulder, et al., 1996. J. Virol. 70:2191-2200.
26. Nicholls, et al., 2003. Lancet 361:1773-1778.
27. Nie, et al., 2004. Biochem. Biophys. Res. Commun. 321:994-1000.
28. Peiris, et al., 2003. N. Engl. J. Med. 349:2431-2441.
29. Peterson, et al., 2006. Curr. Top. Microbiol. Immunol. 303:67-95.
30. Rempel, et al., 2005. J. Virol. 79:4369-4381.
31. Roberts, et al., 2005. J. Virol. 79:5833-5838.
32. Roberts, et al., 2005. J. Virol. 79:503-511.
33. Ryzhikov, et al., 1995. Virol. 140:2243-2254.
34. Shieh, et al., 2005. Hum. Pathol. 36:303-309.
35. Song, et al., 2005. Proc. Natl. Acad. Sci. U.S. A 102:2430-2435.
36. Subbarao, et al., 2004. J. Virol. 78:3572-3577.
37. Subbarao, et al., 2005. Clin. Chem. 51:2333-2340.
39. To, et al., 2004. J. Pathol. 202:157-163.
40. Tseng, et al., 2005. J. Immunol. 174:7977-7985.
41. Tseng, et al., 2005. J. Virol. 79:9470-9479.
42. Tsui, S. K., S. S. Chim, and Y. M. Lo. 2003. N. Engl. J. Med. 349:187-188.
43. Wang, et al., 2006. Clin. Sci. (Lond) 110:393-407.
44. Ward, et al., 2005. Antivir. Ther. 10:263-275.
45. Whitton, et al., 2005. Nat. Rev. Microbiol. 3:765-776.
46. Wong, et al., 2004. Clin. Exp. Immunol. 136:95-103.
47. Xu, et al., 2005. Clin. Infect. Dis. 41:1089-1096.
48. Yamashita, et al., 2005. Biochem. Biophys. Res. Commun. 334:79-85.
49. Yang, et al., 2004. J. Virol. 78:5642-5650.

What is claimed is:

1. An expression vector, comprising:
a constitutive promoter, an intron, a polyadenylation site of rabbit β globin and a nucleotide sequence encoding a human angiotensin converting enzyme-2.

2. The expression vector of claim 1, wherein the constitutive promoter is a lung-specific or epithelium-specific promoter that drives global expression of hACE-2.

3. The expression vector of claim 2, wherein the constitutive promoter is a CAG, CMV, SV40, RSV, PGK, or β-actin promoter.

4. The expression vector of claim 2, wherein the lung-specific promoter is CC10 or SPC promoter.

5. The expression vector of claim 2, wherein the epithelium specific promoter is a Keratin 10, Keratin 14, or Keratin 18 promoter.

6. The expression vector of claim 1, wherein the vector is pCAGGS-ACE.

7. A transgenic mouse expressing human angiotensin converting enzyme-2 (hACE-2), wherein the mouse is derived using the vector of claim 1.

8. The transgenic mouse of claim 7, wherein the transgenic mouse with an inbred genetic background is a mouse with a C57BL/6J or a BALB/cJ background.

9. The transgenic mouse of claim 7, wherein the human ACE-2 is expressed in the spleen, stomach, heart, muscle, brain, kidney, lung, liver, intestine, or testis of the mouse.

10. The transgenic mouse of claim 7, wherein the mouse is Tg-AC12, Tg-AC2², Tg-AC50, Tg-AC63 or Tg-AC70 mouse.

11. The transgenic mouse of claim 10, wherein said transgene is expressed in both the lungs and the brain abundantly.

12. The transgenic mouse of claim 8, wherein said transgene expression is low and restricted to the lungs.

13. The transgenic mouse of claim 11, wherein said mouse is infected by human coronaviruses.

14. The transgenic mouse of claim 13, wherein said coronavirus is a severe acute respiratory syndrome causing corona viral strain (SARS-CoV) or a NL63 strain.

15. The transgenic mouse of claim 14, wherein infection with said coronavirus elicits an acute inflammatory cytokine response.

16. The transgenic mouse of claim 15, wherein said cytokine response comprises expression of IL-1b, IL-6, IL-12p40, IL-12p70, G-CSF, CXCL1 (KC), MIP-1a and MCP-1 in the lungs and the brain of infected transgenic mice.

17. The transgenic mouse of claim 16, wherein said expression of cytokines is delayed and more intense in the brain following infection.

18. The transgenic mouse of claim 14, wherein the mouse infected with the SARS-CoV exhibit clinical manifestations of severe acute respiratory syndrome (SARS) in humans and dies within 8 days.

19. The transgenic mouse of claim 18, wherein said clinical manifestations comprise gross and microscopic abnormalities in the lungs and other organs such as brain.

20. The transgenic mouse of claim 18, wherein high virus titers are detectable in lungs and brains of said mouse.

21. The transgenic mouse of claim 12, wherein said mouse is infected by human coronaviruses.

22. The transgenic mouse of claim 21, wherein said coronavirus is a severe acute respiratory syndrome causing corona viral strain (SARS-CoV) or a NL63 strain.

23. The transgenic mouse of claim 22, wherein high virus titers are detectable in lungs and not in the brain of said mouse.

24. The transgenic mouse of claim 22, wherein the mouse develops the clinical manifestations of severe acute respiratory syndrome (SARS) in humans without any mortality.

25. The transgenic mouse of claim 22, wherein the mouse develops abnormal cardiovascular and renal functions: in maintaining electrolyte homeostasis and impaired reproductive functions.

26. A method of screening for an anti-coronaviral compound, comprising:
administering a pharmacologically effective amount of the compound to the transgenic mouse of claim 9,
infecting the transgenic mouse with the coronavirus; and
monitoring the infected mouse for development of phenotype of disease caused by the coronavirus, wherein absence of said development in the presence of the compound indicates that the compound inhibits the binding of the virus to the angiotensin converting enzyme-2, thereby screening for the anti-coronaviral compound.

27. The method of claim 26, wherein the compound is a protease inhibitor, an interferon, a steroid receptor blocking peptide, a siRNA, or a natural antiviral compound.

28. The method of claim 26, wherein the compound is administered orally, intravenously, intranasally, or by inhalation.

29. The method of claim 26, wherein the coronavirus is a SARS-causing coronaviral strain or a NL63 strain.

30. A method of screening for a compound that inhibits infectivity of a human coronavirus, comprising:
administering pharmacologically effective amount of the compound to the transgenic mouse of claim 11;
infecting the transgenic mouse and a control transgenic mouse with the human coronavirus; and
comparing the incidence of disease caused by the human coronavirus in the mouse subjected to said administration with the incidence of the control mouse lacking said administration, wherein an absence, or a shortening of the disease course, alleviation of symptoms, or the reduction of mortality rate in the mouse subjected to said administration indicates that the compound inhibits the infectivity of the human coronavirus.

31. The method of claim 30, wherein the compound inhibits the infectivity by inhibiting binding of the human coronavirus to angiotensin converting enzyme-2, by eliciting a protective immune response against the human coronavirus or a combination thereof.

32. The method of claim 31, wherein the compound inhibiting the binding of the coronavirus to the angiotensin converting enzyme-2 is a peptide that blocks receptor binding of the virus.

33. The method of claim 31, wherein the compound is administered orally, intravenously, intramuscularly or subcutaneously.

34. The method of claim 31, wherein the compound eliciting the protective immune response against the human coronavirus is an immunogenic compound or reagent effective as a vaccine, wherein the compound or reagent comprises a viral antigen, a peptide, a virus-like particle, an inactivated virus, a live attenuated virus or viral DNA.

35. The method of claim 34, wherein the compound is administered intramuscularly, intranasally or pericutaneously.

36. The method of claim 34, wherein the human coronavirus uses human angiotensin converting enzyme-2 as a receptor for entry.

37. The method of claim 34, wherein the human coronavirus is a SARS-causing coronaviral strain or a NL63 strain.

38. The method of claim 34, wherein the compound is administered concurrent with or prior to the infection of the mouse with the coronavirus.
